(19) United States
(12) Patent Application Publication
(10) Pub. No.: US 2003/0166979 A1
(43) Pub. Date: Sep. 4, 2003

(54) Chemical methods

(76) Inventors: Dainis Dakternieks, Highton (AU); Carl H. Schiesser, Highton (AU); Tamara Perchyonok, Caulfield (AU)

Correspondence Address: Mark D. Moore, Williams, Morgan & Amerson, P.C., Suite 250, 7676 Hillmont, Houston, TX, 77040, US

(21) Appl. No.: 10/263,360

(22) Filed: Oct. 1, 2002

Related U.S. Application Data
(63) Continuation-in-part of application No. 09/917,415, filed on Jul. 27, 2001.
(60) Provisional application No. 60/221,071, filed on Jul. 27, 2000.

(51) Int. Cl. C07C 29/136; C07C 215/08; C07C 319/02; C07C 391/00; C07C 395/00

(52) U.S. Cl. 568/885; 568/61; 562/899; 564/503

(57) ABSTRACT

Disclosed is a method for enantioselectively-reducing a prochiral carbon-centered radical having one or more electron donator groups attached directly to the central prochiral carbon atom of the radical, and/or attached to a carbon atom within 1 to 4 atoms of the central prochiral carbon atom, comprising treating said radical with a chiral non-racemic organotin hydride in the presence of a Lewis acid.

### BACKGROUND OF THE INVENTION

The present application is a continuation-in-part of co-pending U.S. application Ser. No. 09/917,415, filed Jul. 27, 2001, which claims priority to U.S. Provisional Application Serial No. 60/221,071, filed Jul. 27, 2000, the entire contents of each of which is specifically incorporated herein by reference in its entirety.

### FIELD OF THE INVENTION

The present invention relates generally to reductive methods useful in chemical synthesis. In particular, the present invention provides enantioselective reductive methods using chiral organostannanes and Lewis acids.

### DESCRIPTION OF RELATED ART

The scientific literature contains numerous reports of free-radical reactions proceeding with distereocontrol, (see for example, reviews such as Curran, D. P., et al, Stereochemistry of Radical Reactions, VCH, Weinheim, 1995; Smadja, W., et al Synlett., 1994, 1; Porter, N. A., et al, Acc. Chem., Res., 1991, 24, 296; and Sibi, M., et al, Acc. Chem., Res., 1999, 32, 163). However, there are relatively very few examples of free-radical reactions which proceed with genuine enantiocontrol. The majority of the examples that demonstrate enantioselective outcomes involve the use of chiral auxiliaries and, as a result, are actually further examples of diastereo-selectivity in free-radical chemistry.

Of the remaining few reports, the introduction of asymmetry in the substrate has been achieved through the use of chiral Lewis acid mediation (see for example, Guindon, Y., et al, Tetrahedron Lett., 1990, 31, 2845; Guindon, Y., et al, J. Am. Chem. Soc., 1991, 113, 9701 and Renaud, P., et al Angew,. Chem. Int. Ed., 1998, 37, 2563), or by a chiral reagent through the use of chiral ligands on the tin atoms in suitably constructed stannanes (Schumann, H., et al, J. Organomet. Chem. 1984, 265,145; Curran, D. P., et al, Tetrahedron; Asymmetry, 1996, 7, 2417; Blumstein, M., et al, Angew. Chem. Int. Ed., 1997, 36, 235 and Schartzkopf, K., et al, Eur. J. Chem., 1998, 177).

It has now been found that the enantioselectivity of free radical reductions using chiral non-racemic stannanes can be enhanced by the use of an appropriate Lewis Acid.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Improved methods for the enantioselective reduction of prochiral carbon radicals using chiral and achiral Lewis acids in conjunction with chiral non-racemic stannanes have now been developed which result in an enhanced enantioselectivity when compared to the use of the chiral non-racemic stannane alone.

Accordingly, the present invention provides a method for enantioselectively reducing a prochiral carbon centred radical having one or more electron donor groups attached directly to the central prochiral carbon atom of the radical, and/or attached to a carbon atom within 1 to 4 atoms of the central prochiral carbon atom, comprising treating said radical with a chiral non-racemic organotin hydride in the presence of a Lewis acid.

Preferably, the electron donor group is attached directly to the central prochiral carbon atom or to a carbon atom within 1 or 2 atoms of the central prochiral carbon atom.

In a particular embodiment, the invention is directed towards a method of producing optically enhanced α or β-amino acids, by treatment of a prochiral amino acid carbon centred radical with a chiral non-racemic organotin hydride in the presence of a Lewis acid, wherein the central prochiral carbon atom is an α-carbon atom of an α-amino acid or a β-carbon atom of an β-amino acid.

### DESCRIPTION OF ILLUSTRATIVE EMBODIMENTS

As used herein, the term “prochiral carbon centred radical” is a radical of formula R₁R₂R₃C., wherein each R residue is different and is not hydrogen. Accordingly, the central prochiral carbon atom is the carbon atom to which the R residues are attached. Reduction of the prochiral carbon centred radical with a hydrogen atom donor affords the chiral compound R₁R₂R₃CH. The present invention thus relates to the preparation of enantioselectively enhanced chiral compounds.

The prochiral carbon centred radical can be generated from any suitable radical precursor using methods known in the art. Exemplary radical precursors include aryl, e.g., phenyl, selenides; aryl, e.g., phenyl, sulfides; aryl, e.g., phenyl, tellurides; xanthates; thionoformates and Barton esters (see e.g., B. Giese, Radicals in Organic Synthesis—Formation of C—C Bonds (1986) Pergamon Press, Oxford, the contents of which are specifically incorporated herein by reference in its entirety). Particularly suitable radical precursors for generating the prochiral carbon centred radicals for use in the invention are tertiary chiral halosubstrates, i.e., R₁R₂R₃C-halogen, where R₁-R₃ are different and not hydrogen and halogen is chlorine, bromine or iodine, preferably bromine.

The prochiral carbon centred radicals which can be reduced by the methods of the invention include radicals which bear one or more electron donor groups directly on the prochiral central carbon atom and/or attached to a carbon atom α, β, γ, or δ to the central prochiral carbon atom, i.e., within 1, 2, 3 or 4 atoms, preferably within 1 or 2 atoms. Suitable electron donor groups include those containing an electron donor atom such as oxygen, nitrogen, and/or sulfur and which will not be affected by the organotin hydride. One example of an electron donor group is a carbonyl group C(═O), present, as for example, in aldehydes, ketones, carboxy acid, carboxy esters, carboxy amides, anhydrides, lactones, lactams, carbonates, carbamates and thioesters, etc. Other electron donor groups include, thioalkyl groups, amines (unsubstituted or substituted once or twice by, for example, a group selected from alkyl, acyl and aryl), hydroxy groups and ethers (e.g., alkyl and aryl). A preferred electron donor is a carbonyl group. Preferably the carbonyl group is adjacent to, i.e., α- to the chiral carbon to be reduced. Expressed in another way, the prochiral carbon centred radical has at least one electron donor atom within 5 atoms (i.e., 1, 2, 3, 4, or 5) of the central prochiral carbon atom. It will be recognised that some electron donor groups may contain one or more electron donating atoms, e.g., carboxy acid, carboxy ester, thioester, carboxy amide. A prochiral carbon centred radical may also contain more than one electron donating group attached to the central prochiral atom.

Exemplary prochiral carbon centred radicals include those of the formula R₁R₂R₃C., wherein R₁-R₃ are different (and not hydrogen) and are independently selected from alkyl, alkenyl, alkynyl, aryl, heterocyclyl, acyl, amino, substituted amino, carboxy, anhydride, carboxy ester, carboxy amide, lactone, lactam, thioester, formyl, optionally protected hydroxy, thioalkyl, alkoxy, alkenyloxy, alkynyloxy, aryloxy, heterocyclyloxy; or alternatively, any two of R₁-R₃ can together, with the central prochiral carbon atom, form a mono- or poly-cyclic group or fused polycyclic group including as cycloalkyl, cycloalkenyl, cycloalkynyl, a lactone, a lactam, cyclic anhydride, or heterocyclyl and bi-, tri- and tetracyclic fused combinations thererof. At least one of R₁-R₃, or a cyclic group formed by any two of R₁-R₃, contains an electron donor atom within 1 to 5 atoms of the prochiral central carbon atom to be reduced. It will be understood that a radical precursor may contain more than one prochiral radical precursor sites and that reduction may therefore occur at one or more of these sites.

In one preferred embodiment, at least one of R₁-R₃ is an optionally substituted aryl or heteroaryl group. In another preferred embodiment at least one of R₁-R₃ is an optionally substituted alkyl, alkenyl, or alkynyl group. In another embodiment, at least one of R₁-R₃ is a ketone, aldehyde, carboxy acid, carboxy ester, carboxy amide, anhydride, lactone, lactam or thioester, or two of R₁-R₃ together with the central prochiral carbon atom form a cyclic anhydride, lactam or lactone.

Preferred “ketones” have the formula —C(O)—R wherein R can be any residue, having a carbon atom covalently bonded to the carbonyl group, such as alkyl, alkenyl, alkynyl and aryl. An R group may have one or more carbon atoms optionally replaced with one or more heteroatoms to form, for example, heterocyclyl.

Preferred “carboxy esters” have the formula —CO₂R wherein R can be any residue, having a carbon atom covalently bonded to the non-carbonyl oxygen atom, for example, alkyl, alkenyl, alkynyl or aryl. An R group may have one or more carbon atoms optionally replaced with one or more heteroatoms, such that R is for example heterocyclyl.

Preferred “carboxy amides” have the formula CO₂NRR′ wherein R and R′ are independently selected from hydrogen and any residue having a carbon atom covalently bonded to the nitrogen atom such as alkyl, alkenyl, alkynyl or aryl. An R or R′ group may have one or more carbon atoms optionally replaced with one or more heteroatoms to form, for example, heterocyclyl.

Preferred “thioesters” have the formula —C(O)SR wherein R can be any residue having a carbon atom covalently bonded to the sulfur atom, such as alkyl, alkenyl, alkynyl or aryl. An R group may have one or more carbon atoms optionally replaced with one or more heteroatoms to form, for example, heterocyclyl.

Preferred “anhydrides” contain the moiety —C(O)—OC(O)— and may be cyclic or acyclic. Preferred acyclic anhydrides contain the moiety —C(O)—O—C(O)—R wherein R can be any residue, such as alkyl, alkenyl, alkynyl or aryl. An R-group may have one or more carbon atoms optionally replaced with one or more heteroatoms to form, for example, heterocyclyl. Preferred cyclic anhydrides contain the moiety —C(O)—O—C(O)—(CH₂)ₙ— wherein nis≧1, e.g., 1, 2, 3, 4, 5 or 6.

Lactones are cyclic residues containing the moiety —C(O)O—. Preferred “lactones” have the formula —C(O)O—R— wherein-R-can be any residue, having a carbon atom covalently bonded to the non-carbonyl oxygen atom, e.g., alkylene, alkenylene, alkynylene. An R-group may have one or more carbon atoms optionally replaced by one or more heteroatoms. Preferred lactones contain the moiety —C(O)—O—(CH₂)ₙ— wherein n is >2, e.g., 2, 3, 4, 5 or 6.

Lactams are cyclic residues containing the moiety —C(O)—N(R′)—R— wherein R′ can be hydrogen or any hydrocarbon residue such as alkyl, acyl, aryl or alkenyl. —R— can be any hydrocarbon residue having a carbon atom covalently bonded to the nitrogen atom such as alkylene, alkenylene or alkynylene. An R′ or R group may have one or more carbon atoms optionally replaced by one or more heteroatoms. Preferred lactams contain the moiety —C(O)—N(R′)—(CH₂)ₙ— wherein n is >2, eg., 2, 3, 4, 5 or 6.

As used herein, the term “alkyl”, denotes straight chain, branched or cyclic hydrocarbon residues, preferably C₁₋₂₀ alkyl, eg C₁₋₁₀ or C₁₋₆. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2,-trimethylpropyl, 1,1,2-trimethylpropyl, heptyl, 5-methoxyhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propylocytl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like.

Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, “butyl” etc, it will be understood that this can refer to any of straight, branched and cyclic isomers. An alkyl group may be optionally substituted by one or more optional substituents as herein defined. Accordingly, “alkyl” as used herein is taken to refer to optionally substituted alkyl. Cyclic alkyl may refer to monocyclic alkyl or, polycyclic fused or non-fused carbocyclic groups.

The term “alkenyl” as used herein denotes groups formed from straight chain, branched or cyclic hydrocarbon residues containing at least one carbon to carbon double bond including ethylenically mono-, di- or poly-unsaturated alkyl or cycloalkyl groups as previously defined, preferably C₁₋₂₀ alkenyl (eg C₁₋₁₀ or C₁₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substitutents as herein defined. Accordingly, “alkenyl” as used herein is taken to refer to optionally substituted alkenyl. Cyclic alkenyl may refer to monocyclic alkenyl or, polycyclic fused or non-fused alkenyl carbocyclic groups.

As used herein the term “alkynyl” denotes groups formed from straight chain, branched or cyclic hydrocarbon residues containing at least one carbon-carbon triple bond including ethynically mono-, di- or poly-unsaturated alkyl or cycloalkyl groups as previously defined. Unless the number of carbon atoms is specified the term preferably refers to C₁₋₂₀ alkynyl. Examples include ethynyl, 1-propynyl, 2-propynyl, and butynyl isomers, and pentynyl isomers. An alkynyl group may be optionally substituted by one or more optional substitutents as herein defined. Accordingly, “alkynyl” as used herein is taken to refer to optionally substituted alkynyl. Cyclic alkynyl may refer to monocyclic alkynyl or, polycyclic fused or non-fused alkynyl carbocyclic groups.

The terms “alkoxy”, “alkenoxy”, “alkynoxy”, “aryloxy” and “heterocyclyloxy” respectively denote alkyl, alkenyl, alkynyl, aril and heterocylclyl groups as hereinbefore defined when linked by oxygen.

The term “halogen” denotes chlorine, bromine or iodine.

The term “aryl” denotes single, polynuclear, conjugated and fused residues of aromatic hydrocarbon ring systems. Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Aryl may be optionally substituted as herein defined and thus “aryl” as used herein is taken to refer to optionally substituted aryl.

The term “heterocyclic” denotes mono- or polycarbocyclic groups, which may be fused or conjugated, aromatic (heteroaryl) or non-aromatic, wherein at least one carbon atom is replaced by a heteroatom, preferably selected from nitrogen, sulphur and oxygen. Suitable heterocyclic groups include N-containing heterocyclic groups, such as: unsaturated 3 to 6 membered heteromonocyclic groups containing 1 to 4 nitrogen atoms, for example, pyrrolyl, pyrrolinyl, imidazolyl, imidazolinyl, pyrazolyl, pyridyl, pyrimidinyl, pyrazinyl, pyridazinyl, triazolyl or tetrazolyl; saturated 3 to 6-membered heteromonocyclic groups containing 1 to 4 nitrogen atoms, such as, pyrrolidinyl, imidazolidinyl, piperidyl, pyrazolidinyl or piperazinyl; condensed saturated or unsaturated heterocyclic groups containing 1 to 5 nitrogen atoms, such as, indolyl, isoindolyl, indolinyl, isoindolinyl, indolizinyl, isoindolizinyl, benzimidazolyl, quinolyl, isoquinolyl, indazolyl, benzotriazolyl, purinyl, quinazolinyl, quinoxalinyl, phenanthradinyl, phenathrolinyl, phthalazinyl, naphthyridinyl, cinnolinyl, pteridinyl, perimidinyl or tetrazolopyridazinyl; saturated 3 to 6-membered heteromonocyclic groups containing 1 to 3 oxygen atoms, such as tetrahydrofuranyl, tetrahydropyranyl, tetrahydrodioxinyl, unsaturated 3 to 6-membered hetermonocyclic group containing an oxygen atom, such as, pyranyl, dioxinyl or furyl; condensed saturated or unsaturated heterocyclic groups containing 1 to 3 oxygen atoms, such as benzofuranyl, chromenyl or xanthenyl; unsaturated 3 to 6-membered heteromonocyclic group containing 1 to 2 sulphur atoms, such as, thienyl or dithiolyl; unsaturated 3 to 6-membered heteromonocyclic group containing 1 to 2 oxygen atoms and 1 to 3 nitrogen atoms, such as, oxazolyl, oxazolinyl, isoxazolyl, furazanyl or oxadiazolyl; saturated 3 to 6-membered heteromonocyclic group containing 1 to 2 oxygen atoms and 1 to 3 nitrogen atoms, such as, morpholinyl; unsaturated condensed heterocyclic group containing 1 to 2 oxygen atoms and 1 to 3 nitrogen atoms, such as, benzoxazolyl or benzoxadiazolyl; unsaturated 3 to 6-membered heteromonocyclic group containing 1 to 2 sulphur atoms and 1 to 3 nitrogen atoms, such as, thiazolyl, thiazolinyl or thiadiazoyl; saturated 3 to 6-membered heteromonocyclic group containing 1 to 2 sulphur atoms and 1 to 3 nitrogen atoms, such as, thiazolidinyl, thiomorphinyl; and unsaturated condensed heterocyclic group containing 1 to 2 sulphur atoms and 1 to 3 nitrogen atoms, such as, benzothiazolyl or benzothiadiazolyl.

A heterocyclic group may be optionally substituted by an optional substituent as described herein.

The term “acyl” denotes a group containing the moiety C═O (and not being a carboxylic acid, ester or amide or thioester). Preferred acyl includes C(O)—R, wherein R is hydrogen or an alkyl, alkenyl, alkynyl, aryl or heterocyclyl, residue, preferably a C₁₋₂₀ residue. Examples of acyl include formyl; straight chain or branched alkanoyl such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g., phenylacetyl, phenylpropanoyl, phenylbutanoyl, phenylisobutylyl, phenylpentanoyl and phenylhexanoyl) and naphthylalkanoyl (e.g., naphthylacetyl, naphthylpropanoyl and naphthylbutanoyl]; aralkenoyl such as phenylalkenoyl (e.g., phenylpropenoyl, phenylbutenoyl, phenylmethacryloyl, phenylpentenoyl and phenylhexenoyl and naphthylalkenoyl (e.g., naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. Acyl also refers to optionally substituted acyl.

The term “acyloxy” refers to acyl, as herein before defined, when linked by oxygen.

In this specification “optionally substituted” is taken to mean that a group may or may not be further substituted or fused (so as to form a condensed polycyclic group) with one or more groups selected from alkyl, alkenyl, alkynyl, aryl, hydroxy, alkoxy, alkenyloxy, aryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, acyl, acylamino, diacylamino, acyloxy, alkylsulphonyloxy, arylsulphenyloxy, heterocyclyl, heterocycloxy, heterocyclamino, carboalkoxy, carboaryloxy, alkylthio, arylthio, acylthio, cyano, nitro, sulfate and phosphate groups.

Preferred optional substituents include, alkyl, (e.g., C₁₋₆alkyl such as methyl, ethyl, propyl, butyl, cyclopropyl, cyclobutyl, cyclopentyl or cyclohexyl), hydroxyalkyl (eg hydroxymethyl, hydroxyethyl, hydroxypropyl), alkoxyalkyl (eg methoxymethyl, methoxyethyl, methoxypropyl, ethoxymethyl, ethoxyethyl, ethoxypropyl, etc.) alkoxy (e.g., C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted), benzyl (wherein benzyl itself may be further substituted), phenoxy (wherein phenyl itself may be further substituted), benzyloxy (wherein benzyl itself may be further substituted), amino, alkylamino (e.g., C₁₋₆alkyl, such as methylamino, ethylamino, propylamino, etc.), dialkylamino (e.g., C₁₋₆alkyl, such as dimethylamino, diethylamino, dipropylamino), acylamino (e.g., NHC(O)CH₃), phenylamino (wherein phenyl itself may be further substituted), nitro, formyl, —C(O)-alkyl (eg C₁₋₆ alkyl, such as acetyl), O—C(O)-alkyl (e.g., C₁₋₆alkyl, such as acetyloxy), benzoyl (wherein the phenyl group of the benzoyl may itself be further substituted), carbonyl, (i.e., replacement of CH₂ with C═O) CO₂H, CO₂alkyl (e.g., C₁₋₆ alkyl such as methyl ester, ethyl ester, propyl ester, butyl ester), CO₂ phenyl (wherein phenyl itself may be further substituted), CONH₂, CONHphenyl (wherein phenyl itself may be further substituted), CONHbenzyl (wherein benzyl itself may be further substituted), CONHalkyl (e.g., C₁₋₆ alkyl such as methyl ester, ethyl ester, propyl ester, butyl amide), CONHdialkyl (e.g., C₁₋₆alkyl).

As used herein, □heteroatom□ refers to any atom other than a carbon atom which may be a ring-member of a cyclic organic compound. Examples of suitable heteroatoms include nitrogen, oxygen, sulfur, phosphorous, boron, silicon, arsenic, selenium and tellurium.

The reductive methods of the invention are carried out for a time and under conditions sufficient to effect enantioselective reduction of a suitable prochiral radical precursor by hydrogen. Suitable reaction temperatures, solvents and quantities of stannane and initiator for free radical reductions are known in the art (see for example V. T. Perchyonok et al., Tetrahedron. Lett., 1998, 39, 5437 and references cited therein). Preferred solvents include hydrocarbon solvents, eg toluene. The reduction is preferably carried out at temperature less than 0° C., preferably less than about −30° C., more preferably at about −78° C. Preferably, the reagents used and the reaction conditions employed are substantially anhydrous. Exemplary initiators include those which are reactive at these temperatures such as AMBM (Tetrahedron Lett., 1997, 38, 6301); 9-BBN (Tetrahedron Lett., 1998, 39, 5437), 9-alkyl-9-BBN, (e.g., alkyl=ethyl, propyl, butyl, etc.).

Exemplary chiral non-racemic organotin hydrides have the formula L₁L₂L₃SnH wherein L₁-L₃ are ligands, which may be the same or different, and wherein at least one of L₁-L₃ has a chiral centre. Suitable non-chiral ligands include optionally substituted aryl (eg optionally substituted phenyl, and napthyl) and non-chiral alkyl (e.g., butyl). Suitable chiral ligands include menthyl and fused polycyclics such as 3α-cholestane and those derived from cholic acid e.g., 3α-24-norcholanyl and 7α-24-norcholanyl (Schiesser et al., Phosphorus, Sulfur, Silicon and Related Elements, (1999) Vol 150-51, 177).

Examples of organotin hydrides include (1R,2S,5R)-menthyldiphenyltin hydride (a) and its enantiomer (1S,2R,5S)-menthyldiphenyltin hydride (a′), bis[(1R,2S,5R)-menthyl]phenyltin hydride (b) and its enantiomer bis[(1S,2R,5S)-menthyl]phenyltin hydride (b′), tris[(1R,2S,5R)-menthyl]tin hydride (c) and 3a-dimethylstannyl-5α-cholestane (d), which can be prepared in accordance with the procedures described in Dakternieks et al., Organometallics, 1999, 3342-3347.

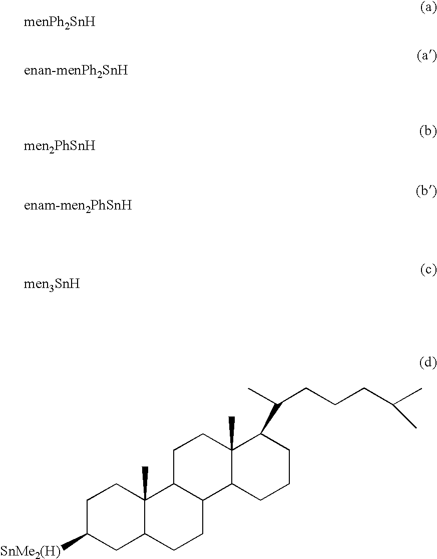

In the above structures,

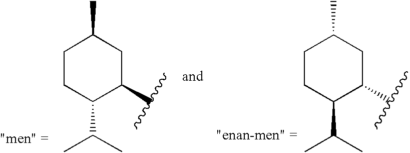

Other suitable organotin hydrides include (e) and (f), which can be prepared by reaction of the appropriate aryl lithium with bis[(1R,2S,5R)-menthyl]phenyltin chloride followed by LiAlH₄ reduction (Dakternieks et al., supra, and Jastrzebski et al, J. Organomet. Chem., 1983, 246, C75 and van Koten et al, Tetrahedron 1989, 45, 569). Other aryl tin hydrides can be made in an analogous manner. Further examples of a suitable organotin hydride include (e) as below, where one of the menthyl groups is replaced by a phenyl group (both diasteroisomers). Other exemplary preferred compounds of the invention include, for example, (g) shown below.

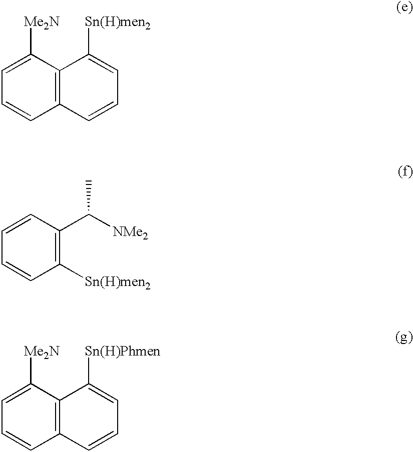

Lewis acids for use with the method of the present invention are compounds which are able to accept an electron pair, i.e., co-ordinate with an electron donor. Suitable Lewis acidic compounds include transition metal complexes, alkaline earth metal compounds and other metal based compounds wherein the metal centre can accept an electron pair. Examples of suitable Lewis acids include AlCl₃, Me₃A₁, BF₃, BBr₃, BCl₃, Ln(OTf)₃, TiCl₄, FeCl₃, ZnCl₂, zirconocene dichloride (herein after referred to as (i)), trialkylborates (RO₃B, wherein each R is an alkyl group which can be the same or different), (S,S)- and (R,R)-(+)-N,N′-bis(3,5-di-tert-butylsalycidene)-1,2-diaminocyclohexamanganese (III) chloride (hereinafter referred to as, (ii) and (iii) respectively) (Jacobson's catalyst, Jacobsen et al., J. Am. Chem. Soc., 1991, 113, 7063).

Preferably, the Lewis acid has a solubility, under the reaction conditions employed, of at least about 0.1 molar equivalents, preferably at least about 0.5 molar equivalents, more preferably at least about 1.0 molar equivalent, most preferably about 2.0 molar equivalents, per prochiral carbon centred radical to be reduced.

Particularly preferred Lewis acids are those which are alkaline earth metal compounds. When used in accordance with the method of the present invention, such compounds surprisingly afford excellent enantioselectivity.

Accordingly, in a further aspect of the present invention there is provided a method for enantioselectively reducing a prochiral carbon centred radical having one or more electron donor groups attached directly to the central prochiral carbon atom of the radical, and/or attached to a carbon atom within 1 to 4 atoms of the central prochiral carbon atom, comprising treating said radical with a chiral non-racemic organotin hydride in the presence of a Lewis acidic alkaline earth metal compound.

Preferably, the alkaline earth metal compound is a Lewis acidic magnesium compound. Examples of suitable Lewis acidic magnesium compounds include MgBr₂, MgI₂, Mg(OAc)₂ and Mg(OTf)₂. It will be appreciated that the above list of magnesium compounds is not exhaustive and that the invention encompasses the use of other Lewis acidic magnesium compounds or combinations thereof.

An increase in the size of the Lewis acid has been found to generally result in an increase in enantioselectivity. However, contrary to this general trend it has been found that the relatively small Lewis acid MgBr₂ surprisingly affords excellent enantioselectivity. In particular, the use of MgBr₂ as a Lewis acid in accordance with the method of the present invention has been found to provide ee values that are significantly greater than those obtained from the use other Lewis acids such as BF₃, Me₃A1, ZnCl₂ and Ln(OTf)₃.

The use of MgBr₂ as a Lewis acid in accordance with the present invention has a further advantage in that it is cheap and readily available.

Accordingly, where the Lewis acid used in accordance with the method of the present invention is a Lewis acidic magnesium compound, the Lewis acidic magnesium compound is preferably MgBr₂.

Those skilled in the art will appreciate that Lewis acids can often be conveniently provided in the form of a Lewis adduct, that is, an adduct formed from a Lewis acid and a Lewis base. In particular, those skilled in the art will appreciate that a Lewis adduct can be used as a convenient source for providing a Lewis acid to a reaction. Accordingly, Lewis acids used in accordance with the present invention may also be provided in the form of a Lewis adduct. For example, Lewis acids such as BF₃, ZnCl₂, and MgBr₂ may be provided and used in the form of their diethylether adducts BF₃.(Et₂O)₂, ZnCl₂.(Et₂O)₂ and MgBr₂.(Et₂O)₂, respectively.

The stannane is preferably used in an amount of about 0.5-1.5 molar equivalents per reductive site on the substrate, i.e., central prochiral carbon atom, more preferably about 1.1 molar equivalents, to effect optimum reductive conversion.

In general, the Lewis acid is preferably used in an amount of about 0.9 to about 2.0 molar equivalents, more preferably in an amount of about 0.9 to about 1.1 molar equivalents, per reductive site on the substrate, i.e., central prochiral carbon atom. In particular, the Lewis acid is preferably used in an amount of about 1.5 molar equivalents, most preferably about 1.0 molar equivalents per reductive site on the substrate, i.e., central prochiral carbon atom. Lesser amounts can be used such as 0.1 or 0.5 molar equivalents although lower enantiomeric excesses (ee's) are usually observed. The addition of higher amounts of Lewis acid can also be used, although this does generally not result in an increase in observed ee's.

When the Lewis acid is an alkaline earth metal compound, it is preferable that the Lewis acid is used in an amount of about 1.5 molar equivalents, more preferably about 2.0 molar equivalents, per prochiral carbon centred radical to be reduced. In particular, when the Lewis acid is a magnesium compound, it is preferable that the Lewis acid is used in an amount of about 1.5 molar equivalents, more preferably about 2.0 molar equivalents, per prochiral carbon centred radical to be reduced.

The stereochemistry of the reduced prochiral carbon centre in the resulting compound can be R or S.

The methods of the invention may be particularly useful in preparing optically enhanced amino acids. Thus, α- or β-carbon centred radicals derived from α- or β-substituted amino acids may be reduced by the methods of the invention to produce optically enhanced amino acids which may be natural or unnatural, including alanine, asparagine, cysteine, glutamine, isoleucine, leucine, methionine, phenylalanine, proline, serine, threonine, tryptophan, tyrosine, valine, aspartic acid, glutamic acid, arginine, histidine, lysine and their homo derivatives. Other examples include α-and β-straight and branched chain alkyl substituted amino acids, α- and β-cycloalkyl substituted amino acids, and α- and β-aryl substituted amino acids

The chiral stannanes for the generation of the prochiral carbon centred radical may also be immobilized onto a solid support, eg a polymeric support, such as pins, beads or wells, for use in the methods of the invention, eg used in combinatorial techniques known in the art.

The invention will now be described with reference to the following non-limiting examples which are included for the purpose of illustrating the invention only and are not to be construed as limiting the generality hereinbefore described.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Reduction of Compounds (1a)-(1d) and (2).

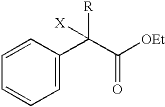

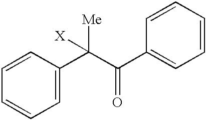

| | | | | |
|---|---|---|---|---|
| 1a. | R = Me, | X = Br | 2. | X = Br |
| 1b. | R = Et, | X = Br | | |
| 1c. | R = cyclopentyl | X = Br | | |
| 1d. | R = Bu^{t}, | X = Br | | |

Compounds la-d and 2 (X=Br) were prepared according to the methods of Metzger et al Angew. Chem., Int., Ed. Engl., 1997, 36, 235 and Curran, et al, Tetrahedron: Asymmetry, 1996, 7, 2417.

Reductions were carried out in toluene at −78° C. The reaction solution comprised the substrate at a concentration of approximately 0.1M, the Lewis acid of choice at about 1.0 molar equivalents, relative to the substrate, and about 1.1 molar equivalents, relative to the substrate, of the stannane. The reaction was initiated using 9-BBN (V. T. Perchyonok et al, Tetrahedron. Lett, 1998, 39, 5437).

Table 1 lists enantioselectivity data for the model substrates 1 and 2 (X═Br) reacting with bis[(1R,2S,5R)-menthyl]phenyltin hydride at −78° C. in toluene in the absence of any additives and in the presence of 1 molar equivalent, relative to the substrate, of BF₃, zirconocene dichloride (i), (S,S)-(−)- or (R,R)-(+)-N,N′-bis(3,5-di-tert-butylsalycidene)-1,2diaminocyclohexanemanganese (III) chloride (ii) or (iii) respectively. The results show that an increase in the size of the Lewis acid generally results in an increase in enantioselectivity. See for example entries 3-5, 8-10, 13-15 and 18-20 in Table 1 where the addition of (ii) or (iii) provided greater ee values than the use of (i), with, in many cases, (i) providing a greater enantiomeric excess (ee) compared to BF₃.

All conversions gave the (S) product. The conversion data shown in the following Tables was measured by GC. Isolated yields for some compounds are shown in parenthesis.

TABLE 1
Enantioselectivities observed for reactions involving
bis[(1R,2S,5R)-menthyl] phenyltin hydride (b) in toluene at −78° C.

| Entry | Substrate | Lewis Acid | Ee (%) | Conversion (%) |
|---|---|---|---|---|
| 1 | 1a | None | 2 | 80 |
| 2 | 1a | BF₃ | 32 | 64 |
| 3 | 1a | (i) | 36 | 58 |
| 4 | 1a | (ii) | 60 | 81 |
| 5 | 1a | (iii) | 55 | 59 |
| 6 | 1b | None | 4 | 81 |
| 7 | 1b | BF₃ | 20 | 68 |
| 8 | 1b | (i) | 46 | 52 |
| 9 | 1b | (ii) | 86 | 75 (71)* |
| 10 | 1b | (iii) | 84 | 69 |
| 11 | 1c | None | 9 | 81 |
| 12 | 1c | BF₃ | 30 | 79 |
| 13 | 1c | (i) | 35 | 74 |
| 14 | 1c | (ii) | 80 | 82 (71)* |
| 15 | 1c | (iii) | 78 | 75 |
| 16 | 1d | None | 6 | 82 |
| 17 | 1d | BF₃ | 10 | 76 |
| 18 | 1d | (i) | 60 | 68 |
| 19 | 1d | (ii) | 80 | 72 |
| 20 | 1d | (iii) | 83 | 52 |
| 21 | 2 | None | 16 | 81 |
| 22 | 2 | BF₃ | 12 | 69 |
| 23 | 2 | (i) | 52 | 92 |
| 24 | 2 | (ii) | 52 | 76 |
| 25 | 2 | (iii) | 50 | 60 |

*Isolated yield

### Example 2

Table 2 lists the effect that different organotin hydrides have on the observed enantioselectivities at −78° C. for reactions involving Lewis acids, (i), (ii) and (for one example) (iii). Reactions were carried out in accordance with the procedure described in Example 1. It should be noted that the achiral stannane, tributyltin hydride, reacts with Id (X═Br) in the presence of Lewis acids (i) and (ii) to afford Id (X=H) with 0 and 8% ee, respectively.

TABLE 2
Enantioselectivities observed for reactions involving zirconocene
dichloride (i) and (S,S)-(-)-N,N'-bis(3,5-di-tert-butylsalycidene)-
1,2-di-aminocyclohexanemanganese(III) chloride (ii) and its enantiomer
(iii) in toluene at −78° C.

| Entry | Substrate | Lewis Acid | Stannane | Ee (%) | Conversion (%) |
|---|---|---|---|---|---|
| 1 | 1a | (i) | (a) | 36(S) | 59 |
| 2 | 1a | (i) | (e) | 38(S) | 77 |
| 3 | 1a | (i) | (d) | 60(S) | 82 |
| 4 | 1a | (ii) | (a) | 60(S) | 82 |
| 5 | 1a | (ii) | (f) | 90(S) | 73 (68)* |
| 6 | 1a | (ii) | (d) | 34(S) | 58 |
| 7 | 1b | (i) | (a) | 42(S) | 51 |
| 8 | 1b | (i) | (e) | 52(S) | 79 |
| 9 | 1b | (i) | (d) | 54(S) | 54 |
| 10 | 1b | (ii) | (b) | 70(S) | 78 |
| 11 | 1b | (ii) | (f) | 72(S) | 68 |
| 12 | 1b | (ii) | (d) | 62(S) | 67 |
| 13 | 1b | (iii) | (b′) | 86(R) | 72 |
| 14 | 1c | (ii) | (f) | 96(S) | 75 (67)* |
| 15 | 1d | (i) | Bu₃SnH | 0(−) | — |
| 16 | 1d | (i) | (a) | 58(S) | 63 |
| 17 | 1d | (i) | (e) | 62(S) | 87 |
| 18 | 1d | (i) | (d) | 76(S) | 96 |
| 19 | 1d | (ii) | Bu₃SnH | 8(S) | — |
| 20 | 1d | (ii) | (a) | 72(S) | 74 |
| 21 | 1d | (ii) | (f) | 80(S) | 76 |
| 22 | 1d | (ii) | (d) | 82(S) | 72 (68)* |
| 23 | 2 | (i) | (a) | 50(S) | 68 |
| 24 | 2 | (1) | (e) | 56(S) | 62 |
| 25 | 2 | (i) | (d) | 42(S) | 79 |
| 26 | 2 | (ii) | (a) | 58(S) | 81 |
| 27 | 2 | (ii) | (e) | 46(S) | 85 |
| 28 | 2 | (ii) | (f) | 62(S) | 74 |
| 29 | 2 | (ii) | (d) | 52(S) | 72 |

*Isolated yield

### Example 3

Reduction of Compounds 1b, 3 and (4a-d).

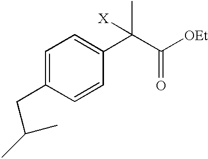

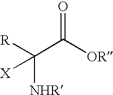

| | | | | |
|---|---|---|---|---|
| 3. X = Br | 4a. | R = Et, | R″ = Me | X = Br |
| | 4b. | R = tert-Bu, | R″ = Bn | X = Br |
| | 4c. | R = Ph, | R″ = Me | X = Br |
| | 4d. | R = Bn | R″ = Me | X = Br |

R′ = COCF₃
Bn = Benzyl

Compounds 3 and 4a-d (X═Br) were prepared as follows:

Preparation of Compound (3)

Racemic ibuprofen (0.5 g, 2.42 mmol) and bromine (0.425 g, 1.1 eq, 2.66 mmol) were heated under reflux and PBr₃ (0.67 g, 1.03 eq, 2.49 mmol) slowly added to the reaction mixture. The reaction mixture was further heated at 65-70° C. until the evolution of HBr had ceased (approx. 3 hours). The reaction mixture was then distilled to remove residual HBr and low boiling impurities. A 1:1 mixture of ethanol/dichloromethane (5 ml) was slowly added followed by a small amount of H₂SO₄ (approx 1 drop) and the reaction mixture was heated at reflux for a further 2 hours. The remaining solvent was removed in vacuo to afford (3) in sufficient purity for further use (0.265 g).

General Procedure for Preparation of Compound (4a, 4c-d, X═Br).

The N-trifluoroacetyl amino acid methyl esters (4a, 4c-d, X═H) (100 mg), prepared as described below, were dissolved in carbon tetrachloride (5 ml) and N-bromosuccinimide (NBS) (1 equiv) was added. The mixture was irradiated (under reflux) by a 250W tungsten lamp for 45 minutes. The solid was removed by filtration and the solvent removed in vacuo to afford (4a, 4c-d, X═Br) in quantitative yield and of sufficient purity for further use.

Preparation of Compound (4b)

A mixture of racemic tert-leucine (0.2 g), dry methanol (0.5 ml), triethylamine (0.3 ml) and methyl trifluoroacetate (0.16 ml) was allowed to stir at room temperature for 15 hours. Removal of methanol in vacuo afforded the triethylammonium salt of N-trifluoroacetyltertleucine as a crystalline mass which was dissolved in dry DMF (0.5 ml). Triethylamine (0.14 ml) and benzyl chloride (0.35 g) were added and the mixture allowed to stir at room temperature for 40 hours. The resulting mixture was poured into ethyl acetate, washed with H₂O, 5% HCl, sat. NaHCO₃ and brine. The organic layer was dried (MgSO₄) and the solvent removed in vacuo to obtain the crude product as a light yellow oil. Pure N-trifluoroacetyl-tert-leucine benzyl ester was obtained as a pale oil after flash chromatography (96:4 hexane: ethyl acetate) in 65% yield.

N-bromosuccinimide (NBS) (61 mg) was added to a solution of N-trifluoroacetyl-tert-leucine benzyl ester (100 mg) in carbon tetrachloride (5 ml). The mixture was irradiated (under reflux) by a 250W tungsten lamp for 45 minutes. The solid was removed by filtration and the solvent removed in vacuo to afford (4b) in quantitative yield and of sufficient purity for further use.

General Reduction Procedure

Reductions were carried out in toluene at −78° C. The reaction solution comprised the substrate at a concentration of approximately 0.1 M, about 1.3 molar equivalents, relative to the substrate, of the required stannane and the Lewis acid of choice in either about 1.0 or 2.0 molar equivalents, relative to the substrate, depending on the Lewis acid chosen (see Tables 3 and 4). Reactions were initiated with Et₃B/O₂. Reactions were carried out until TLC analysis indicated the absence of starting material (ca. 1-2 h) at which time the reaction mixtures were examined by chiral-phase gas chromatography (CG) and the percentage conversion and enantiomeric ratios determined by integration of the signals corresponding to the mixture of reduced compounds 1 and 2 (X═H) against an internal standard (either octane or undecane). -Reduced compounds 1 and 2 (X═H) were identified by comparison of their GC retention times with those of the authentic compounds. Gas Chromatographic analyses of the reaction mixtures were carried out using a chiral trifluoroacteylated y-cyclodextrin (Chiraldex™ G-TA, 30 mm×0.25 mm) capillary column purchased from Alltech. The absolute configuration of the dominant isomer in each case was assigned by comparison with the GC retention times of the (S)-products 1 and 2, prepared and resolved following literature procedures (Campbell, A., et al, J. Chem. Soc., 1946, 25; Aaron, C., et al, J. Org. Chem.; and Elhafez, F. A. A., et al, J. Am. Chem. Soc., 1952, 74, 5846), and the (S)-products 3 and 4, prepared by the following procedures:

Preparation of (S)-Ibuprofen Methyl Ester (3, X═H)

A solution of commercially available (S)-ibuprofen (2 g, 9.66 mmol) in thionyl chloride (10 ml) was heated at reflux until the evolution of gas ceased. The excess thionyl chloride was removed in vacuo and solution of ethanol (5 ml) in dichloromethane (10 ml) was added and refluxing continued for further 2 hour. The mixture was cooled and the solvent removed in vacuo to give (3, X═H) as colourless oil (1.61 g, 71%) and of sufficient purity for further use.

General Procedure for Preparation of (4a, 4c-d, X═H).

Commercially available racemic or S-2-aminobutyric acid, phenylglycine or phenylalanine (1 g) was stirred overnight at room temperature in dry methanol (3 ml) containing trimethylsilyl chloride (Me₃SiCl) (3-5 equivalents). The solvent was removed in vacuo to obtain the corresponding amino acid methyl ester hydrochloride as a white solid with no need for further purification.

Triethylamine (1.1 eq) was added to a stirred solution of the amino acid methyl ester hydrochloride and methyl tryfluoroacetate (1.5 equivalents) in dry methanol (10 ml). The reaction was heated under reflux for 12 hours, after which the solvent was removed and resulting residue redissolved in ether (20 ml). The solution was washed with sat, ammonium chloride, dried (MgSO₄) and the ether removed in vacuo to obtain the corresponding required N-trifluoroacetyl amino acid methyl ester of sufficient purity for further use.

Preparation of (S)-N-trifluoroacetyl-tert-leucine Benzyl Ester (4b, X═H)

A mixture of (S)-tert-leucine (0.2 g), dry methanol (0.5 ml), triethylamine (0.3 ml) and methyl trifluoroacetate (0.16 ml) was allowed to stir at room temperature for 15 hours. Removal of methanol in vacuo afforded the triethylammonium salt of N-trifluoroacetyl-tert-leucine as a crystalline mass which was dissolved in dry DMF (0.5 ml). Triethylamine (0.14 ml) and benzyl chloride (0.35 g) were added and the mixture allowed to stir at room temperature for 40 hours. The resulting mixture was poured into ethyl acetate, washed with H₂O, 5% HCl, sat. NaHCO₃ and brine. The organic layer was dried (MgSO₄) and the solvent removed in vacuo to obtain the crude product as a light yellow oil. Pure (S)-N-trifluoroacetyl-tert-leucine benzyl ester was obtained as a pale oil after flash chromatography (96:4 hexane: ethyl acetate) in 65% yield.

Table 3 lists enantioselectivity data for the reduction of substrates 3, 4a-d (X═Br) with bis[(1R,2S,5R)-menthyl]phenyltin hydride (b) at −78° C. in toluene in the absence of any additives and in the presence of about 2.0 molar equivalents of MgBr₂.Et₂O, relative to the substrate.

Table 4 lists enantioselectivity data for the reduction of substrates 3 and 4a-c (X═Br) with (1R,2S,5R)-menthyldiphenyltin hydride (a), (1S,2R,5S)-menthyldiphenyltin hydride (a′), bis[(1R,2S,5R)-menthyl]phenyltin hydride (b), bis[(1S,2R,5S)-menthyl]phenyltin hydride (b′) or tris[(1R,2S,5R)-menthyl]tin hydride (c) at −78° C. in toluene in the absence of any additives and in the presence of about 1.0 molar equivalents of BF₃, Me₃Al, ZnCl₂.(Et₂O)₂, Ln(OTf)₃ or about 2.0 molar equivalents of MgBr₂.(Et₂O)₂, relative to the substrate.

The results in Tables 3 and 4 show that MgBr₂ is a particularly effective Lewis acid when used in accordance with the method of the present invention. See for example entries 2, 6, 8 and 10 where the use of MgBr₂.(Et₂O)₂ affords ee values greater than 96%. Also, the superiority of MgBr₂.(Et₂O)₂ as a Lewis acid is shown in Table 4 where the ee values obtained from its use are significantly greater than those obtained from the use other Lewis acids such as BF₃.(Et₂O)₂, Me₃Al, ZnCl₂.(Et₂O)₂ and Ln(OTf)₃.

The results shown in Tables 3 and 4 also demonstrate that by the selective choice of stannane, the absolute configuration of the reduced substrate can be effectively controlled. For example, bis[(1R,2S,5R)-menthyl]phenyltin hydride (b) and bis[(1S,2R,5S)-menthyl]phenyltin hydride (b′) react with substrate 3 (X=Br) to provide (S)- and (R)-ibuprofan methyl ester, respectively, with 96% ee in each case (compare entry 2, Table 3 with entry 6, Table 4).

TABLE 3
Enantioselectivities observed for reactions involving racemic
organohalides with bis((1R,2S,5R)-menthyl)phenyltin hydride (b) in
toluene at −78° C., using about 2.0 molar equivalents of MgBr₂·(Et₂O)₂,
relative to the substrate

| Entry | Substrate | Lewis Acid | Ee (%) | Conversion (%) |
|---|---|---|---|---|
| 1 | 3 | none | 31(S) | * |
| 2 | 3 | MgBr₂.(Et₂O)₂ | 96(S) | 63 |
| 3 | 4a | None | 0 | * |
| 4 | 4a | MgBr₂.(Et₂O)₂ | 10 | * |
| 5 | 4b | None | 18(S) | 45 |
| 6 | 4b | MgBr₂.(Et₂O)₂ | 99(S) | 65 |
| 7 | 4c | None | 5(S) | 70 |
| 8 | 4c | MgBr₂.(Et₂O)₂ | 99(R) | 78 |
| 9 | 4d | None | 6(S) | * |
| 10 | 4d | MgBr₂.(Et₂O)₂ | 99(R) | 81 |

*Not determined

TABLE 4
Enantioselectivities observed for reactions involving the broad range
of Lewis acids and stannanes in toluene at −78° C. MgBr₂·(Et₂O)₂
was used in about 2.0 molar equivalents, and all other Lewis acids were
used in about 1.0 molar equivalent, relative to the substrate

| Entry | Substrate | Lewis acid | Stannane | Ee(%) | Conversion (%) |
|---|---|---|---|---|---|
| 1 | 3 | BF₃.(Et₂O)₂ | (b) | 66(S) | 75 |
| 2 | 3 | none | (a) | 10(S) | * |
| 3 | 3 | MgBr₂.(Et₂O)₂ | (a) | 80(S) | 70 |
| 4 | 3 | none | (a′) | 8(R) | 60 |
| 5 | 3 | MgBr₂.(Et₂O)₂ | (a′) | 80(R) | 68 |
| 6 | 3 | MgBr₂.(Et₂O)₂ | (b′) | 96(R) | 80 |
| 7 | 4b | none | (b′) | 20(R) | 60 |
| 8 | 4b | MgBr₂.(Et₂O)₂ | (b′) | 96(R) | 58 |
| 9 | 4c | BF₃.(Et₂O)₂ | (b) | 13(5) | * |
| 10 | 4c | Me₃Al | (b) | 12 (S) | * |
| 11 | 4c | ZnCl₂.(Et₂O)₂ | (b) | 14(S) | * |
| 12 | 4c | Ln(OTf)₃ | (b) | 14(S) | * |
| 13 | 1b | MgBr₂.(Et₂O)₂ | (c) | 71(S) | 65 |
| 14 | 3 | MgBr₂.(Et₂O)₂ | (c) | 62(5) | * |
| 15 | 3 | none | (c) | 5(S) | * |
| 16 | 4b | MgBr₂.(Et₂O)₂ | (c) | 96(R) | 60 |
| 17 | 4b | none | (c) | 18(R) | * |

*Not determined

A typical experimental procedure for the reduction reactions conducted in Example 3 is described below:

Reduction of N-TFA-bromo-tertleucinemethyl Ester (4b):

MgBr₂.Et₂O (0.121 g, 0.478 mmol) was added to dry toluene (0.2 ml) and the mixture allowed to stir for 20 minutes under nitrogen. The bromoester (4b) (0.08 g, 0.234 mmol) in dry toluene (0.1 ml) was added slowly to the reaction mixture and allowed to stir at RT for a further 10 min prior to cooling to −78° C. After stirring at −78° C. for 45 min, men₂PhSnH (b) (0.2 g, 0.42 mmol) in toluene (0.25 ml) was added slowly, followed by Et₃B (0.1 ml of 1 M solution in THF) and 02 was introduced. The reaction mixture was stirred at this temperature for a further 4 hours. The mixture was quenched with H₂O (5 ml) and extracted with Et₂O (2×). The organic layer was then dried with MgSO₄ and excess of solvent removed in vacuo to afford the crude product as a light yellow oil. Further purification of the product (column chromatography 96:4 hexane/ethyl acetate) yielded (S)-N-TFA-tertleucinemethyl ester as a colourless oil (65%), a^{13.7}d(C=0.4, CHCl₃)=6. 2. ¹-(NMR) CDCl₃: δ 7.2 (5H, m, Ar—H), 5.3 (2H, q, —CH₂), 4.5 (1H, d, —CH—), 1.0 (9H, s, tert-But). HPLC analysis indicated an enantioselectivity in excess of 99%.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below.

What is claimed is:

1. A method for enantioselectively reducing a prochiral carbon centred radical having one or more electron donor groups attached directly to the central prochiral carbon atom of the radical, and/or attached to a carbon atom within 1 to 4 atoms of the central prochiral carbon atom, comprising treating said radical with a chiral non-racemic organotin hydride in the presence of a Lewis acid.

2. The method of claim 1, wherein the electron donor group is attached directly to the central prochiral carbon atom or to a carbon atom within 1 or 2 atoms of the central prochiral carbon atom.

3. The method of claim 1, wherein the prochiral carbon centred radical is a prochiral amino acid carbon centred radical wherein the central prochiral carbon atom is an α-carbon atom of an α-amino acid or a β-carbon atom of an β-amino acid.

4. The method of claim 1, wherein the prochiral carbon centred radical is generated from a radical precursor selected from the group consisting of: aryl selenides, aryl sulphides, aryl tellurides, xanthates, thionoformates, Barton esters and tertiary chiral halosubstrates.

5. The method of claim 1, wherein the electron donor group is a carbonyl group.

6. The method of claim 1, wherein the organotin hydride is selected from the group consisting of:

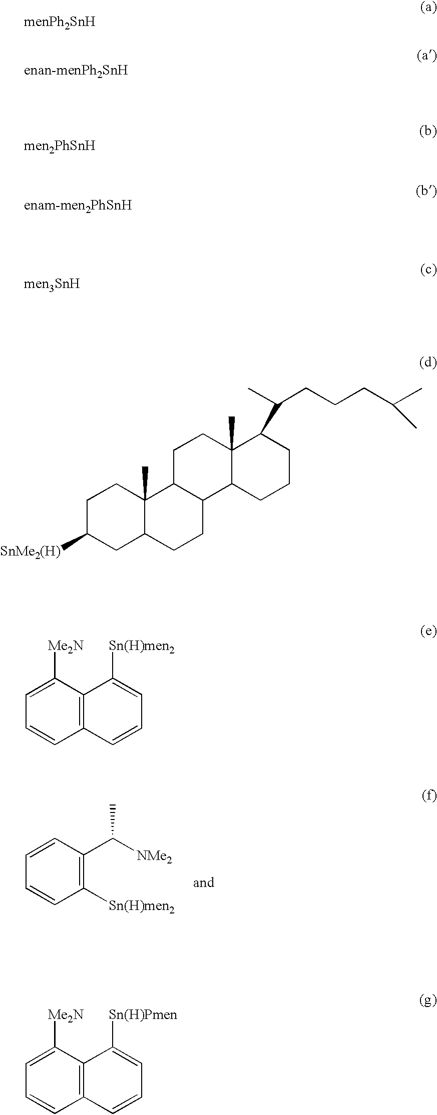

7. The method of claim 1, wherein the Lewis acid is selected from the group consisting of: AlCl₃, Me₃A₁, BF₃, BBr₃, BCl₃, Ln(OTf)₃, TiCl₄, FeCl₃, ZnCl₂, zirconocene dichloride, trialkylborates and (S,S)- and (R,R)-(+)-N,N′-bis (3,5-di-tert-butylsalycidene)-1,2-diaminocyclohexamanganese (III) chloride.

8. The method of claim 1, wherein the Lewis acid is provided in the form of a Lewis adduct.

9. The method of claim 8, wherein the Lewis adduct is selected from the group consisting of BF₃.(Et₂O)₂ and ZnCl₂.(Et₂O)₂.

10. The method of claim 1, wherein the Lewis acid is used in an amount of about 0.9 to about 2.0 molar equivalents per prochiral carbon centred radical to be reduced.

11. The method of claim 1, wherein the Lewis acid is used in an amount of about 0.9 to about 1.1 molar equivalents per prochiral carbon centred radical to be reduced.

12. The method of claim 1, wherein the organotin hydride is used in an amount of about 0.5 to about 1.5 molar equivalents per prochiral carbon centred radical to be reduced.

13. The method of claim 1, wherein the organotin hydride is immobilized onto a solid support.

14. The method of claim 1, wherein the Lewis acid has a solubility, under the reaction conditions employed, of at least about 0.1 molar equivalents per prochiral carbon centred radical to be reduced.

15. A method for enantioselectively reducing a prochiral carbon centred radical having one or more electron donor groups attached directly to the central prochiral carbon atom of the radical, and/or attached to a carbon atom within 1 to 4 atoms of the central prochiral carbon atom, comprising treating said radical with a chiral non-racemic organotin hydride in the presence of a Lewis acidic alkaline earth metal compound.

16. The method of claim 15, wherein the electron donor group is attached directly to the central prochiral carbon atom or to a carbon atom within 1 or 2 atoms of the central prochiral carbon atom.

17. The method of claim 15, wherein the prochiral carbon centred radical is a prochiral amino acid carbon centred radical wherein the central prochiral carbon atom is an α-carbon atom of an α-amino acid or a β-carbon atom of an β-amino acid.

18. The method of claim 15, wherein the prochiral carbon centred radical is generated from a radical precursor selected from the group consisting of: aryl selenides, aryl sulphides, aryl tellurides, xanthates, thionoformates, Barton esters and tertiary chiral halosubstrates.

19. The method of claim 15, wherein the electron donor group is a carbonyl group.

20. The method of claim 15, wherein the organotin hydride is selected from the group consisting of:

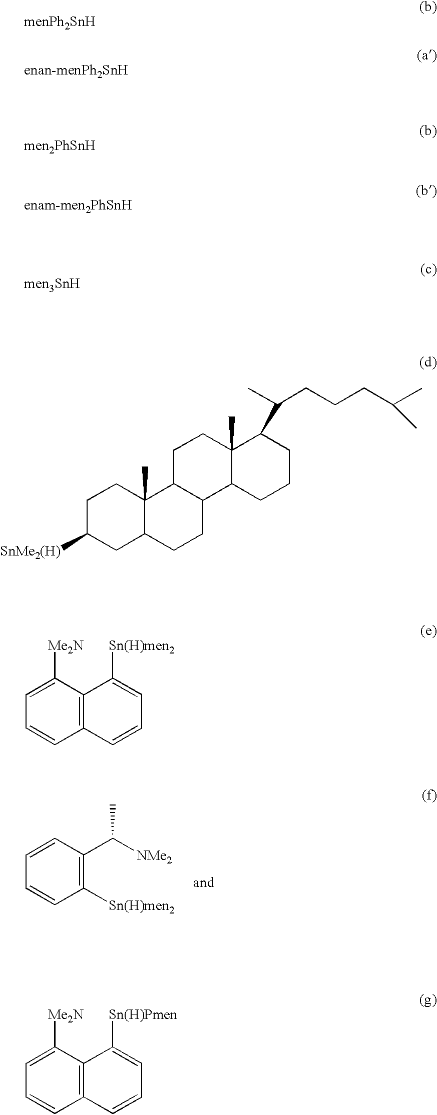

21. The method of claim 15, wherein the alkaline earth metal compound is a Lewis acidic magnesium compound.

22. The method of claim 21, wherein the Lewis acidic magnesium compound is selected from the group consisting of MgBr₂, MgI₂, Mg(OAc)₂, Mg(OTf)₂.

23. The method of claim 21, wherein the Lewis acidic magnesium compound has a solubility, under the reaction conditions employed, of at least about 0.1 molar equivalents per prochiral carbon centred radical to be reduced.

24. The method of claim 21, wherein the Lewis acidic magnesium compound has a solubility, under the reaction conditions employed, of about 2.0 molar equivalents per prochiral carbon centred radical to be reduced.

25. The method of claim 21, wherein the Lewis acidic magnesium compound is provided in the form of a Lewis adduct.

26. The method of claim 21, wherein the Lewis acidic magnesium compound is MgBr₂.

27. The method of claim 25, wherein the Lewis adduct is MgBr₂.(Et₂O)₂.

28. The method of claim 15, wherein the Lewis acidic alkaline earth metal compound is used in an amount of about 0.9 to about 2.0 molar equivalents per prochiral carbon centred radical to be reduced.

29. The method of claim 15, wherein the Lewis acidic alkaline earth metal compound is used in an amount of about 2.0 molar equivalents per prochiral carbon centred radical to be reduced.

30. The method of claim 21, wherein the Lewis acidic magnesium compound is used in an amount of about 0.9 to about 2.0 molar equivalents per prochiral carbon centred radical to be reduced.

31. The method of claim 21, wherein the Lewis acidic magnesium compound is used in an amount of about 2.0 molar equivalents per prochiral carbon centred radical to be reduced.

32. The method of claim 15, wherein the organotin hydride is used in an amount of about 0.5 to about 1.5 molar equivalents per prochiral carbon centred radical to be reduced.

33. The method of claim 15, wherein the organotin hydride is immobilized onto a solid support.
